(19) United States
(12) Patent Application Publication
(10) Pub. No.: US 2002/0037863 A1
(43) Pub. Date: Mar. 28, 2002

(54) Novel anthelmintic combinations

(76) Inventor: Timothy G. Geary, Kalamazoo, MI (US)

Correspondence Address: Andrew M. Solomon, Pharmacia & Upjohn Company, Global Intellectual Property, 301 Henrietta Street, Kalamazoo, MI, 49001, US

(21) Appl. No.: 09/827,661

(22) Filed: Apr. 6, 2001

Related U.S. Application Data
(60) Provisional application No. 60/195,394, filed on Apr. 7, 2000.

(51) Int. Cl. A61K 31/7048; A61K 31/407; A61K 31/365

(52) U.S. Cl. 514/28; 514/409; 514/450

(57) ABSTRACT

An anthelmintic composition comprising:

(a) one or more active ingredients which is a member from the family of macrocyclic lactones; and

(b) one or more active ingredients which is a member from the family of spirodioxepinoindoles is provided. The composition is used to treat or prevent parasitic diseases.

### BACKGROUND OF THE INVENTION

1. Field of the Invention

The present invention relates to novel anthelmintic compositions in general, and, more specifically, compositions containing at least one member from the family of macrocyclic lactones and at least one member from the family of spirodioxepinoindoles as active ingredients.

2. Technology Description

Control of parasitic infections in human and animal populations remains an important global endeavor. The causative organisms may be categorized as endoparasitic members of the classes Nematoda, Cestoidea and Trematoda or phylum Protozoa, or as ectoparasitic members of the phylum Arthropoda. The former comprises infections of the stomach, intestinal tracts, lymphatic system, tissues, liver, lungs, heart and brain. Examples include trichinosis, lymphatic filariasis, onchocerciasis, schistosomiasis, leishmaniasis, trypanosomiasis, giardiasis, coccidiosis and malaria. The latter ectoparasites include lice, ticks, mites, biting flies, fleas and mosquitoes. These often serve as vectors and intermediate hosts to endoparasites for transmission to human or animal hosts. While certain helminthiases can be treated with known drugs, evolutionary development of resistance necessitates a further search for improved efficacy in next generation anthelmintic agents.

The control of ectoparasites, such as fleas, ticks, biting flies and the like, has long been recognized as an important aspect of human and animal health regimens. Traditional treatments were topically applied, such as the famous dips for cattle, and indeed such treatments are still in wide use. The more modern thrust of research, however, has been towards compounds which can be administered orally or parenterally to the animals and which will control ectoparasitic populations by poisoning individual parasites when they ingest the blood of a treated animal.

The control of endoparasites, especially intestinal parasites, has also been an important aspect of human and animal health regimens.

Although a number of ectoparasiticides and endoparasiticides are in use, these suffer from a variety of problems, including a limited spectrum of activity, the need for repeated treatment and, in many instances, resistance by parasites. The development of novel endo- and ectoparasiticides is therefore essential to ensure safe and effective treatment of a wide range of parasites over a long period of time.

The milbemycins and avermectins are a group of macrolide anthelmintics and insecticides which have been prepared by the cultivation of microorganisms and are described in inter alia GB-A-1,390,336, J. Antibiotics 29(3), 76-14 to 76-16 and 29 (6), 76-35 to 76-42, GB-A-2 170 499, EP-A-O 073 660 and EP-A-0 204 421. Further anthelmintically active milbemycins and avermectins are described in GB-A-2 176 180, EP-A-0 212 867, EP-A-0 237 339, EP-A-0 241 146, EP-A-0 214 731, EP-A-0 194 125, EP-A-0 170,006, and U.S. Pat. No. 4,285,963. Ivermectin is described in U.S. Pat. No. 4,199,569.

The avermectins are a family of closely related compounds produced by Streptomyces avermitilis and other microbes or by synthetic or semi-synthetic means. Members of the avermectin (C-076) family include other derivatives of pentacyclic 16-membered lactones, primarily A₁ₐ, A₂ₐ, B₁ₐ, B₂ₐ as well as minor components A_{1b}, A_{2b}, B_{1b}, B_{2b}, all of which share to some degree activity as antiparasitics and acaricides. Ivermectin has been marketed for treatment of various helminth intestinal parasites and heartworm in animals and for onchocerciasis (river blindness) in humans. The broad spectrum of activity of the avermectins makes them attractive candidates for treatment of a variety of endo- and ectoparasites.

Resistance to the anthelmintic activity of macrolide lactones such as ivermectin has spread widely among trichostrongyloid parasites of sheep, especially in the species Haemonchus contortus (see Conder G A and Campbell W C, Advances in Parasitology, volume 35, pages 1-84, 1995; Sangster N C, International Journal for Parasitology, volume 29, pages 115-124, 1999). There are currently no available methods to selectively reduce the frequency of anthelmintic resistance alleles in parasite populations using chemical treatments.

The marcfortines are known compounds, see Journal of the Chemical Society Chemical Communications, 601-602 (1980) for Marcfortine A and Tetrahedron Letters, 22, 1977-1980 (1981) for Marcfortines B and C. These compounds are fungal metabolites of Penicillium roqueforti. The marcfortines are structurally related to the paraherquamides, which are also known compounds.

The paraherquamides are disclosed in Tetrahedron Letters, 22, 135-136 (1981), and Journal of Antibiotics, 44, 492-497 (1991). U.S. Pat. Nos. 4,866,060 and 4,923,867 disclose the use of the marcfortines A, B, and C, and certain derivatives thereof as useful for the treatment and prevention of parasitic diseases in animals.

WO 92/22555 (published 23 Dec. 1992) generically describes a marcfortine or paraherquamide derivative (i.e. partial formula (III) substituted at position 14 with methyl or methyl and hydroxy). WO 91/09961 (published 11 Jul. 1991) discloses various derivatives of marcfortine and paraherquamide, and 12a-N-oxides thereof.

International Publication WO 92/22555 (published Dec. 23, 1992) generically discloses 14.alpha-hydroxymarcfortine compounds and a process that uses the 14-hydroxy-14-methylmarcfortine compounds for the production of antiparasitic drugs.

2-deoxyparaherquamide and marcfortine derivatives are described in U.S. Pat. Nos. 5,750,695 and 5,703,078.

Recent publications have shown that various strains of several trichostrongyloid parasites resistant to macrocyclic lactones such as ivermectin have an increased susceptibility to spirodioxepinoindoles such as paraherquamide (see Gill J H and Lacey E, International Journal for Parasitology, volume 28, pages 863-877, 1998). This increased sensitivity was characteristic of strains that had evolved resistance in the field, as opposed to those in which selection had been accomplished in the laboratory. Furthermore, the increased sensitivity of ivermectin-resistant strains to paraherquamide has only been characterized in the larval stages (Gill and Lacey, op. cit.). However, the larval stages are not commonly targets for chemotherapy, and the sensitivity of adult stages of these parasites to the combination is unknown. It is well known that the potency and activity of many compounds differ between larval and adult stages of trichostrongyloid parasites. Thus, activity of drugs or drug combinations against larval stages of trichostrongyloid parasites cannot be used to predict activity against adult stages, especially activity in a host animal.

Despite the above teachings, there still exists a need in the art for using chemical additives to specifically reduce the frequency of alleles encoding macrocyclic lactone resistance proteins in adult stages of trichostrongyloid populations, thus maintaining and restoring to utility the macrocyclic lactones for trichostrongyloid control.

### BRIEF SUMMARY OF THE INVENTION

In accordance with the present invention, a novel composition of matter which is capable of specifically reducing the frequency of alleles encoding macrocyclic lactone resistance proteins in trichostrongyloid populations, thus maintaining and restoring to utility the macrocyclic lactones for trichostrongyloid control, is provided. The composition contains at least one member from the family of macrocyclic lactones and at least one member from the family of spirodioxepinoindoles as active ingredients.

A first embodiment of the present invention provides an anthelmintic composition comprising:

(a) one or more active ingredients which is a member from the family of macrocyclic lactones; and

(b) one or more active ingredients which is a member from the family of spirodioxepinoindoles; and, optionally, a pharmaceutically effective carrier.

In particularly preferred embodiments, the active ingredient from component (a) is ivermectin, moxidectin, doramectin, eprinomectin, selamectin or milbemycin oxime; and the active ingredient from component (b) is paraherquamide, 2-deoxyparapherquamide, marcfortine or 14-hydroxymarcfortine.

Another embodiment of the present invention comprises a process for the treatment or prevention of parasitic diseases in mammals, plants or agricultural crops comprising the step of administering to the mammal, plant or crop an effective amount of the above composition.

In preferred embodiments, the mammal is either a food animal, farm animal or companion animal.

A further embodiment of the present invention comprises the use of the above-described composition to prepare a medicament for the treatment or prevention of parasitic diseases in mammals.

Yet another embodiment of the present invention comprises the above-described composition for use as a medicament.

A final embodiment of the present invention comprises a method for reducing the frequency of macrocyclic lactone-resistant individuals in populations of trichostrongyloid nematodes comprising the step of treating such populations with an effective amount of the above-described composition.

An object of the present invention is to provide novel anthelmintic compositions which can be broadly used against parasites which are typically resistant to macrocyclic lactones.

Still another object of the present invention is to provide a method for preventing or treating parasitic diseases in mammals by using a novel composition.

A further object of the present invention is to provide a method for producing a medicament using a novel composition.

These, and other objects, will readily be apparent to those skilled in the art as reference is made to the detailed description of the preferred embodiment.

### DETAILED DESCRIPTION OF THE PREFERRED EMBODIMENT

In describing the preferred embodiment, certain terminology will be utilized for the sake of clarity. Such terminology is intended to encompass the recited embodiment, as well as all technical equivalents which operate in a similar manner for a similar purpose to achieve a similar result.

The present invention is directed to the prevention and treatment of parasitic attack on host animals and provides a new tool for the control of parasitic organisms. In particular, the present invention provides a method of controlling parasites by administering a novel anthelmintic composition that includes:

(a) one or more active ingredients which is a member from the family of macrocyclic lactones; and

(b) one or more active ingredients which is a member from the family of spirodioxepinoindoles.

The first class of compounds are the macrocyclic lactones. These materials are known in the art and have achieved great commercial success as anthelmintics. Examples of such materials are disclosed in GB-A-2 176 180, EP-A-0 212 867, EP-A0 237 339, EP-A-0 241 146, EP-A-0 214 731, EP-A-0 194 125, EP-A-0 170,006, U.S. Pat. Nos. 4,285,963, 4,199,569 and 5,637,703. To the extent necessary for completion, these documents are expressly incorporated by reference.

Preferred groups of this first class of compounds are the avermectins. Of this group, the most preferred compound is ivermectin, which is commercially sold by Merck and Co. under any of the following names: CARDOMEC, EQVALAN, HEARTGARD30, IVOMEC, IVOMEC-F, MECTIZAN, STROMECTOL or ZIMECTERIN. It is believed that the biological action of the avermectins is associated with the disruption of specific glutamate-gated chloride ion channel systems in the affected organisms.

A second class of closely related compounds is the milbemycins. The major differences separating the avermectins and milbemycins are the presence of a disaccharide (oleandrosyl-oleandrose) unit at the C-13 position of the avermectin macrolactone and an acyloxy or hydroxy group at C-22 in the spiroketal portion of the milbemycins.

Specific examples of groups of compounds which are either avermectins or milbemycins include the following: ivermectin, moxidectin, doramectin, eprinomectin, selamectin or milbemycin oxime, with ivermectin being especially preferred.

In practice, the amount of the macrocyclic lactone compound to be administered ranges from about 0.001 to 10 mg. per kg. of animal body weight, such total dose being given at one time or in divided doses over a relatively short period of time such as 1-5 days. Excellent control of such parasites is obtained in animals by administering from about 0.025 to 0.5 mg. per kg. of body weight in a single dose. Repeat treatments are given as required to combat re-infections and are dependent upon the species of parasite and the husbandry techniques being employed. The techniques for administering these materials to animals are known to those skilled in the veterinary field.

The second class of compound which forms part of the inventive composition are the spirodioxepinoindoles. These compounds are discussed in greater detail in the following publications: U.S. Pat. Nos. 4,866,060, 4,923,867, 5,750,695, 5,703,078, WO 92/22555 and WO 91/09961. To the extent necessary for completion, these documents are expressly incorporated by reference.

In particular, the preferred members of the second class of compounds are either the marcfortines, the paraherquamides, or derivatives thereof. Specifically preferred compounds include, but are not limited to paraherquamide, 2-deoxyparapherquamide, marcfortine, 14-hydroxymarcfortine or 14-hydroxy, 15-methylmarcfortine. Particularly preferred is 2-deoxyparapherquamide. The structure of this molecule is shown in Formula XXX of U.S. Pat. No. 5,750,695, where n=1, and its synthesis is described in Example 37 of this patent. In addition, the instant invention is expressly intended to cover the compounds and derivatives from marcfortines A, B and C.

In practice, the amount of the spirodioxepinoindole compound to be administered ranges from about 0.05 to 20 mg. per kg. of animal body weight, such total dose being given at one time or in divided doses over a relatively short period of time such as 1-5 days. Excellent control of such parasites is obtained in animals by administering from about 0.1 to 10.0 mg. per kg. of body weight in a single dose. Repeat treatments are given as required to combat re-infections and are dependent upon the species of parasite and the husbandry techniques being employed. The techniques for administering these materials to animals are known to those skilled in the veterinary field.

For use as an antiparasitic agent in animals the inventive composition may be administered internally either orally or by injection, or topically as a liquid drench or as a shampoo. These compositions may be administered orally in a unit dosage form such as a capsule, bolus or tablet. The drench is normally a solution, suspension or dispersion of the active ingredients usually in water together with a suspending agent such as bentonite and a wetting agent or like excipient. Generally, the drenches also contain an antifoaming agent. Drench formulations generally contains from about 0.01 to 10% by weight of each active compound. Preferred drench formulations may contain from 0.05 to 5.0% of each active by weight. The capsules and boluses comprise the active ingredients admixed with a carrier vehicle such as starch, talc, magnesium stearate, or di-calcium phosphate.

Where it is desired to administer the inventive composition in a dry, solid unit dosage form, capsules, boluses or tablets containing the desired amount of active compounds usually are employed. These dosage forms are prepared by intimately and uniformly mixing the active ingredient with suitable finely divided diluents, fillers, disintegrating agents and/or binders such as starch, lactose, talc, magnesium stearate, vegetable gums and the like. Such unit dosage formulations may be varied widely with respect to their total weight and content of the antiparasitic agent depending upon factors such as the type of host animal to be treated, the severity and type of infection and the weight of the host.

When the active composition is to be administered via an animal feedstuff, it is intimately dispersed in the feed or used as a top dressing or in the form of pellets which may then be added to the finished feed or optionally fed separately. Alternatively, the antiparasitic compositions of the present invention may be administered to animals parenterally, for example, by intraruminal, intramuscular, intratracheal, or subcutaneous injection in which event the active ingredients are dissolved or dispersed in a liquid carrier vehicle. For parenteral administration, the active materials are suitably admixed with an acceptable vehicle, preferably of the vegetable oil variety such as peanut oil, cottonseed oil and the like. Other parenteral vehicles such as organic preparation using solketal, propylene glycol, glycerol formal, and aqueous parenteral formulations are also used, often in combination in various proportions. Still another carrier which can be selected is either N-methylpyrrolidone or 2-pyrrolidone and mixtures of the two. This formulation is described in greater detail in U.S. Pat. No. 5,773,442. To the extent necessary for completion, this patent is expressly incorporated by reference. The active compound or compounds are dissolved or suspended in the parenteral formulation for administration; such formulations generally contain from 0.005 to 5% by weight of each active compound.

In a particularly preferred embodiment, the carrier contains propylene glycol (1-99 percent by weight of the carrier) and glycerol formal (99-1 percent by weight of the carrier), with the relative amounts being 60% propylene glycol and 40% glycerol formal.

The present compositions may also be useful in yet another method in which the same active agents as above defined are employed as a “feed through larvicide.” In this method, the compound is administered to a vertebrate animal, especially a warm-blooded animal, in order to inhibit parasitic organisms which live in the feces of the animal. Such organisms are typically insect species in the egg or larval stage.

The inventive compositions are primarily useful as antiparasitic agents for the treatment and/or prevention of helminthiasis in all mammals, and preferably food animals and companion animals such as cattle, sheep, deer, horses, dogs, cats, goats, swine, and poultry. They are also useful in the prevention and treatment of parasitic infections of these animals by ectoparasites such as ticks, mites, lice, fleas and the like. They are also effective in the treatment of parasitic infections of humans. In treating such infections the inventive compositions may be used individually or in combination with each other or with other unrelated antiparasitic agents.

The exact dosage and frequency of administration of the inventive compositions depend on many factors, including (but not limited to) the severity of the particular condition being treated, the age, weight, and general physical condition of the particular patient (human or animal), and other medication the patient may be taking. These factors are well known to those skilled in the art, and the exact dosage and frequency of administration can be more accurately determined by measuring the concentration of the inventive composition in the patient's blood and/or the patient's response to the particular condition being treated.

The active ingredients of inventive composition may be administered in the same physical form or concomitantly according to the above-described dosages and in the above-described delivery vehicles. The dosages for each active component can be measured separately and can be given as a single combined dose or given separately. They may be given at the same or at different times as long as both actives are in the subject at one time over a 24-hour period. Concomitant or concurrent administration means the patient takes one active within about 5 minutes of taking the other. Because the goal is to provide rapid symptomatic relief to the subject, in most cases when treatment is started the two actives would be administered to the patient close in time and typically concomitantly; thereafter, the timing of each active's administration may not be as important.

The inventive compositions may also be used to combat agricultural pests that attack crops either in the field or in storage. The inventive compositions are applied for such uses as sprays, dusts, emulsions and the like either to the growing plants or the harvested crops. The techniques for applying the inventive compositions in this manner are known to those skilled in the agricultural arts.

Accordingly, it can be seen that the present methods can be utilized for protection against a wide range of parasitic organisms. Further, it should be noted that protection is achieved in animals with existing parasitic infections by eliminating the existing parasites, and/or in animals susceptible to attack by parasitic organisms by preventing parasitic attack. Thus, protection includes both treatment to eliminate existing infections and prevention against future infestations.

Representative parasitic organisms include the following:

Platyhelminthes:

Trematoda such as

Clonorchis

Echinostoma

Fasciola hepatica (liver fluke)

Fasciola gigantica

Fascioloides magna

Fasciolopsis

Metagonimus

Paragonimus

Schistosoma spp.

Nemathelminthes:

Ancylostoma

Angiostrongylus

Anisakis

Ascaris

Brugia

Bunostomum

Cooperia

Cyathostomum

Cylicocyclus

Dictyocaulus (lungworm)

Dipetalonema

Dirofilaria (heartworm)

Dracunculus

Elaeophora

Gaigeria

Globocephalus urosubulatus

Haemonchus

Metastrongylus (lungworm)

Muellerius (lungworm)

Necator americanus

Nematodirus

Oesophagostomum

Onchocerca

Ostertagia

Parascaris

Protostrongylus (lungworm)

Setaria

Stephanofilaria

Syngamus

Teladorsagia

Toxascaris

Toxocara

Trichinella

Trichostrongylus

Uncinaria stenocephala

Wuchereria bancrofti

Arthropoda:

Crustacea:

Argulus

Caligus

Arachnida:

Amblyomma americanum (Lone-star tick)

Amblyomma maculatum (Gulf Coast tick)

Argas persicus (fowl tick)

Boophilus microplus (cattle tick)

Demodex bovis (cattle follicle mite)

Demodex canis (dog follicle mite)

Dermacentor andersoni (Rocky Mountain spotted fever tick)

Dermacentor variabilis (American dog tick)

Dermanyssus gallinae (chicken mite)

Ixodes ricinus (common sheep tick)

Knemidokoptes gallinae (deplumming mite)

Knemidokoptes mutans (scaly-leg mite)

Otobius megnini (ear tick)

Psoroptes equi (scab mite)

Psoroptes ovis (scab mite)

Rhipicephalus sanguineus (brown dog tick)

Sarcoptes scabiei (mange mite)

Insecta:

Aedes (mosquito)

Anopheles (mosquito)

Culex (mosquito)

Culiseta (mosquito)

Bovicola bovis (cattle biting louse)

Callitroga hominivorax (blowfly)

Chrysops spp. (deer fly)

Cimex lectularius (bed bug)

Ctenocephalis canis (dog flea)

Ctenocephalis fells (cat flea)

Culicoides spp. (midges, sandflies, punkies, or no-see-ums)

Damalinia ovis (sheep biting louse)

Dermaobia spp. (warble fly)

Dermatophilus spp. (fleas)

Gasterophilus haemorrhoidalis (nose hot fly)

Gasterophilus intestinalis (common horse hot fly)

Gasterophilus nasalis (chin fly)

Glossina spp. (tsetse fly)

Haematobia irritans (horn fly, buffalo fly)

Haematopinus asini (horse sucking louse)

Haematopinus eurysternus (short nosed cattle louse)

Haematopinus ovilius (body louse)

Haematopinus suis (hog louse)

Hydrotaea irritans (head fly)

Hypoderma bovis (bomb fly)

Hypoderma lineatum (heel fly)

Linognathus ovillus (body louse)

Linognathus pedalis (foot louse)

Linognathus vituli (long nosed cattle louse)

Lucilia spp. (maggot fly)

Melophagus ovinus (sheep ked)

Oestrus ovis (nose hot fly)

Phormia regina (blowfly)

Psorophora

Reduviid bugs (assassin bug)

Simulium spp. (black fly)

Solenopotes capillatus (little blue cattle louse)

Stomoxys calcitrans (stable fly)

Tabanus spp. (horse fly)

Parasitic organisms which live in feces are typically the egg and larval stages of insects such as:

Musca domestica (housefly)

Musca autumnalis (face fly)

Haematobia spp. (horn fly, buffalo fly and others).

While not wishing to be bound to any specific scientific theory, it is believed that the combination of a paraherquamide/marcfortine derivative with a macrocyclic lactone anthelmintic is able to specifically reduce the frequency of alleles encoding macrocyclic lactone resistance proteins in trichostrongyloid populations, thus maintaining and restoring to utility the macrocyclic lactones for trichostrongyloid control. The enhanced potency of the paraherquamide/marcfortine class of anthelmintics against macrocyclic lactone-resistant nematodes will selectively remove said resistant nematodes from the population of parasites. It is further believed that except for the above-described combination, the combination of other dissimilar anthelmintic agents does not necessarily provide the desired reduction.

The invention is described in greater detail by the following non-limiting example.

### EXAMPLE 1

0.1 to 20 parts of ivermectin and 0.5 to 90 parts of 2-deoxyparaherquamide are dissolved in 600 parts of propylene glycol and 400 parts of glycerol (formal). The composition is administered to an animal to treat and/or prevent parasitic diseases. Alternatively, the ivermectin and 2-deoxyparaherquamide actives may be separately dissolved in independent vehicles and each vehicle is then applied to the animal to be treated.

Having described the invention in detail and by reference to the preferred embodiments thereof, it will be apparent that modifications and variations are possible without departing from the scope of the appended claims.

What is claimed is:

1. An anthelmintic composition comprising:
(a) one or more active ingredients which is a member from the family of macrocyclic lactones; and
(b) one or more active ingredients which is a member from the family of spirodioxepinoindoles.

2. The composition according to claim 1 further comprising a pharmaceutically effective carrier.

3. The composition according to claim 2 wherein said active of component (a) is either from the avermectin or milbemycin class of compounds.

4. The composition according to claim 3 wherein said active of component (a) is selected from the group consisting of ivermectin, moxidectin, doramectin, eprinomectin, selamectin and milbemycin oxime and mixtures thereof.

5. The composition according to claim 4 wherein said active of component (a) is ivermectin.

6. The composition according to claim 1 wherein said active of component (b) is either from the marcfortine or paraherquamide classes of compounds or derivatives thereof.

7. The composition according to claim 6 wherein said active of component (b) is selected from the group consisting of paraherquamide, 2-deoxyparapherquamide, marcfortine and 14-hydroxymarcfortine 14-hydroxy, 15-methylmarcfortine and mixtures thereof.

8. The composition according to claim 7 wherein said active of component (b) is 2-deoxyparaherquamide or paraherquamide.

9. The composition according to claim 1 wherein component (a) and component (b) are maintained in the same delivery vehicle.

10. The composition according to claim 1 wherein component (a) and component (b) are maintained in different delivery vehicles.

11. An anthelmintic composition consisting essentially of ivermectin, 2-deoxyparaherquamide or paraherquamide, and a pharmaceutically effective carrier.

12. A process for the treatment or prevention of parasitic diseases in mammals comprising the step of administering to the mammal an effective amount of a composition comprising:
(a) one or more active ingredients which is a member from the family of macrocyclic lactones; and
(b) one or more active ingredients which is a member from the family of spirodioxepinoindoles;
in a pharmaceutically effective carrier.

13. The process according to claim 12 wherein said mammal is selected from the group consisting of humans, cattle, sheep, horses, deer, dogs, cats, goats, swine, and poultry.

14. The process according to claim 12 wherein said method of administration is either orally, by injection or topically.

15. The process according to claim 12 wherein about 0.001 to about 10 mg of active from component (a) and about 0.05 to about 20 mg of active from component (b) per kg of mammal are administered.

16. The process according to claim 15 wherein said active of component (a) is selected from the group consisting of ivermectin, moxidectin, doramectin, eprinomectin, selamectin and milbemycin oxime and mixtures thereof; and wherein said active of component (b) is selected from the group consisting of paraherquamide, 2-deoxyparapherquamide, marcfortine, 14-hydroxymarcfortine and 14-hydroxy, 15-mehtylmarcfortine and mixtures thereof.

17. The process according to claim 12 wherein component (a) and component (b) are maintained in the same delivery vehicle.

18. The process according to claim 12 wherein component (a) and component (b) are maintained in different delivery vehicles.

19. A process for the treatment or prevention of parasitic diseases in plants or agricultural crops comprising the step of administering to the plants or agricultural crops an effective amount of a composition comprising:
(a) one or more active ingredients which is a member from the family of macrocyclic lactones; and
(b) one or more active ingredients which is a member from the family of spirodioxepinoindoles;
in an effective carrier.

20. A method for reducing the frequency of macrocyclic lactone-resistant individuals in populations of trichostrongyloid nematodes comprising the step of treating such populations with an effective amount of a composition comprising:
(a) one or more active ingredients which is a member from the family of macrocyclic lactones; and
(b) one or more active ingredients which is a member from the family of spirodioxepinoindoles;
in a pharmaceutically effective carrier.
